(19) United States
(12) Patent Application Publication
(10) Pub. No.: US 2018/0105802 A1
(43) Pub. Date: Apr. 19, 2018

(54) Viral Targeting with Knottins

(71) Applicant: The Regents of the University of California, Oakland, CA (US)

(72) Inventors: Corey Raffel, San Francisco, CA (US); Sangeet Lal, Berkeley, CA (US)

(21) Appl. No.: 15/783,468

(22) Filed: Oct. 13, 2017

Related U.S. Application Data
(60) Provisional application No. 62/408,735, filed on Oct. 15, 2016.

(51) Int. Cl. C12N 7/00; C07K 14/00; A61K 35/768; A61K 48/00

(52) U.S. Cl. CPC C12N 7/00; C07K 14/00; C12N 2760/18432; A61K 48/005; C12N 2760/18443; A61K 35/768

(57) ABSTRACT

The use of targeting knottin polypeptides provides a means of selectively infecting target cells presenting species bound by the knottins. The targeted viruses may be used as cytotoxic agents, for example, as oncolytic viral therapeutics. Alternatively, the knottin-targeted viruses may be used to transform target cells in a gene therapy or immunotherapy context. An effective retargeted measles virus directed to integrins is demonstrated.

### CROSS-REFERENCE TO RELATED APPLICATIONS

This application claims the benefit of priority to U.S. Provisional Application Ser. No. 62/408,735 entitled “Viral targeting with knottins,” filed Oct. 15, 2016, the contents which are hereby incorporated by reference.

### STATEMENT REGARDING FEDERALLY SPONSORED RESEARCH OR DEVELOPMENT

This invention was made with government support under grant no. CA097257, awarded by the National Institutes of Health. The government has certain rights in the invention.

### REFERENCE TO SEQUENCE LISTING, A TABLE, OR A COMPUTER PROGRAM LISTING COMPACT DISK APPENDIX

This application is submitted with a computer readable sequence listing, submitted herewith via EFS as the ASCII text file named: “UCSF036NP_SL.txt”, file size approximately 9,903 bytes, created on Oct. 12, 2017 and hereby incorporated by reference in its entirety.

### BACKGROUND OF THE INVENTION

There is an ongoing effort to develop engineered viruses to ablate tumors or deliver genes. However, to date, the efficacy of such engineered viruses has been suboptimal. Effective viral infection of target cells has been limited by low infection rates and/or undesirable infection of non-target cells.

For example, the use of replication-competent oncolytic viruses could potentially provide an effective and selective treatment for various cancers. For example, retargeted measles virus has been investigated as a potential therapy for brain tumors. Animal safety studies have demonstrated no evidence of toxicity from injection of measles virus into the brain or cerebral spinal fluid. The use of oncolytic measles virus is being investigated in the treatment of various cancers such as glioblastoma, medulloblastoma and atypical teratoid/rhabdoid tumor.

One of the advantages of using measles virus is the ease with which the virus can be retargeted to cell surface antigens of choice. However, the production of such viruses has proven laborious, as the receptor binding motif used to target the virus is typically a single chain antibody. Antibodies suffer from significant practical limitations, including expensive and complicated recombinant production, poor penetration of solid tumors, and the difficulty of site-specifically incorporating modifications.

Accordingly, there remains a need in the art for novel and effective means of retargeting viruses to treat cancer and for other applications such as gene delivery.

### SUMMARY OF THE INVENTION

Provided herein are novel methods and compositions for the treatment of cancer and delivery of genes, among other applications. The scope of the invention encompasses novel engineered viruses which employ cysteine knot, or knottin, polypeptides. The use of knottins enables efficient and selective targeting of engineered viruses to target cells with high rates of infection. The engineered viruses of the invention provide a means for treating various cancers and other conditions, as well as for the delivery of genes to target cells. The use of knottins enables effective targeting of selected cell types with limited off-target infection.

### BRIEF DESCRIPTION OF THE FIGURES

FIG. 1 is a diagram depicting the genomic organization of a modified knottin-targeted measles virus. N=nucleoprotein, P=phosphoprotein, M=matrix protein, F=fusion protein, H=hemagglutinin, and Knottin denotes the knottin targeting moiety. Two point mutations resulting in the substitutions Y481A and R533A, which eliminate binding of the H-protein to its normal receptors, CD46 and SLAM, respectively, are denoted by stars.

FIGS. 2A, 2B, 2C, 2D and 2E depict infection and killing of various cancer cell lines by knottin-targeted measles virus. The graphs depict the number of live cells (as a percentage of live cells in untreated controls) observed 96 hours post-infection at various multiplicities of infection (MOI). The graphs depict data from at least 3 independent experiments and the error bars show standard error of the mean, with *P value <0.05. FIG. 2A depicts killing of U87 glioblastoma cells. FIG. 2B depicts killing of SF8628 DIPG cells. FIG. 2C depicts killing of D-283 medulloblastoma cells. FIG. 2D depicts killing of Vero monkey kidney cells. FIG. 2E depicts killing of MDAMB-435 melanoma cells. FIG. 2F depicts killing of various cell lines by MV-CKPint and MV-GFP, as described in Example 1, at an MOI of 1.

### DETAILED DESCRIPTION OF THE INVENTION

The scope of the invention encompasses engineered viruses comprising a knottin moiety, wherein the knottin moiety confers selective infection of a target cell type. The target cell will exclusively or highly express (relative to non-target cells) a target binding moiety that is selectively bound by the knottin with high affinity. The engineered viruses of the invention may be utilized in various applications, including the oncolytic targeting of tumor cells and gene therapy uses. The various elements of the invention are described in detail next.

Virus. A first element of the invention is a virus. The virus may be any virus suitable for infection of the target cell type, including RNA and DNA viruses. In one implementation of the invention, the engineered virus comprises an enveloped virus. Enveloped viruses express envelope glycoproteins that enable the infection of host cells by mediating fusion between the viral envelope and host cell membranes. For many enveloped viruses, the endoplasmic reticulum (ER) of the host cell is used to support viral entry, replication, and/or assembly. The inventors of the present disclosure have advantageously discovered that the incorporation of functional knottin proteins into replicating viral particles is enhanced by ER processing. Proper folding of knottin proteins in the ER allows the engineered viruses of the invention to efficiently infect target cells. Exemplary envelope virus classes include measles viruses, herpes viruses, lentiviruses, alpha viruses, pox viruses, and vaccinia viruses. Exemplary viruses include, for example, the herpes simplex virus, Newcastle-disease virus and vesicular stomatitis virus.

In an alternative embodiment, the engineered virus of the invention is not an enveloped virus. For example, the virus may comprise an adenovirus or an adeno-associated virus. In one embodiment, the virus is a pseudotyped or chimeric virus.

The engineered viruses of the invention may comprise viruses that do not normally cause disease or other negative effects in the infected host. Alternatively, the virus may be an attenuated virus, i.e. a virus which has mutated or which has been engineered or otherwise treated such that it does not support pathogenic infection of healthy or non-target cells.

An exemplary virus which may be used in the practice of the invention is the measles virus. In one embodiment, the measles virus is an Edmonston vaccine strain measles virus. This strain has been successfully used for decades in immunizations, with over one billion doses delivered and an exemplary safety record. The Edmonston virus is attenuated yet replication competent, and has previously been adapted for experimental oncolytic applications. The Edmonston virus comprises two point mutations in the hemagglutinin protein, resulting in the substitutions Y481A and R533A, which eliminates binding of the H-protein to its normal receptors, CD46 and SLAM, respectively, rending the virus attenuated.

Knottin Targeting Moiety. The scope of the invention encompasses engineered viruses expressing one or more knottin polypeptides. Also known as inhibitor cysteine knot, the knottin motif comprises three disulfide bridges which confer a unique “knot” structure to the polypeptide. Naturally occurring knottins are structurally diverse and have various functions, acting as protease inhibitors, antimicrobials, toxins, and in other roles. Knottins can selectively bind complementary species with high selectivity and affinity, often with nanomolar or picomolar binding constants. Knottins can be engineered to bind diverse target species, and large libraries of knottins with a range of specificities are known. Due to theft disulfide core, knottins are extremely resistant to thermal unfolding, chemical denaturation, and proteolytic degradation. Knottins are generally not immunogenic.

A knottin, as used herein, will refer to knottin and knottin-like polypeptides, as known in the art. For example, in one implementation, a knottin comprises a polypeptide of 15-200 amino acids, for example, 20-60 amino acids, comprising at least six cysteine residues, wherein the cysteine residues form three disulfide bridges. In order, from the amino to carboxyl terminus of the polypeptide sequence, the six cysteines may be referred to as the first, second, third, fourth, fifth, and sixth cysteines. A first disulfide bridge is formed between the first and fourth cysteines, a second disulfide bridge is formed between the second and fifth cysteines, and a third disulfide bridge is formed between the third and sixth cysteines, wherein the third disulfide bridge passes through a loop formed by the first and second disulfide bridges, forming the “knot.” In some knottins, the third disulfide bridge is not present, and knottins, as used herein, will include such polypeptides having two disulfide bridges.

The knottin moiety will be used to direct the engineered virus of the invention to preferentially infect a class of cells, referred to herein as “target cells.” The knottin moiety will be selected based on its specific or preferential binding to a target binding moiety. The target binding moiety will be a species that is present exclusively or is more abundant (compared to non-target cells), on the surface of the target cells. The target binding moiety may be any composition of matter, including proteins, protein domains and motifs, carbohydrates, lipids, chemical entities and other compositions of matter. In one embodiment, the target binding moiety is an extracellular protein domain. In one embodiment, the target binding moiety is a membrane protein.

Exemplary knottins include those described in: U.S. Pat. No. 8,536,301, entitled “Engineered Integrin Binding Peptides,” by Cochran et al.; U.S. Pat. No. 9,206,237, entitled “Cysteine Knot Proteins that Bind alpha-v-beta 6 Integrins,” by Kimura et al.; and U.S. Pat. No. 8,778,888, entitled “Cysteine knot peptides binding to alpha IIb beta 3 integrins and methods” by Cochran et al.; Gracy and Chiche, 2011, “Structure and modeling of knottins, a promising molecular scaffold for drug discovery,” Curr Pharm Des 17(38): 4337-50; and Ackerman et al., 2014, “Cysteine-knot peptides: emerging tools for cancer imaging and therapy,” Expert Rev Proteomics 11(5):561-72. In one embodiment, the knottin is Chrorotoxin, or a variant thereof, for example as described in United States Patent Application Publication Number 20130195760, by Olson et al., entitled “Chlorotoxin variants, conjugates, and methods for their use.” Additional knottins may be generated for specificity to a particular target binding moiety using methods known in the art, for example by sequence randomization, phage display, and amplification selection techniques.

Viral Retargeting. The engineered virus of the invention will comprise one or more knottin moieties. For convenience, the description provided herein will make reference to retargeted viruses comprising a single knottin entity. However, it will be understood that the scope of the invention extends to engineered viruses comprising two or more knottins of different sequences, for example to improve specificity or infectivity or to broaden the range of cells targeted by the engineered virus.

In the engineered virus of the invention, the knottin moiety is configured to facilitate viral docking, fusion, and infection of target cells by the binding of the knottin to the target binding moiety on the target cell surface. The knottin moiety may be configured by various transductional targeting methods known in the art to direct the engineered virus to infect the target cells.

In a preferred implementation, the knottin is expressed in an extra-viral configuration. “Extra-viral,” as used herein, means the knottin moiety is present on the surface of the virus, for example, on the surface of the viral envelope in the case of an enveloped virus, such that it may contact host cells. For example, the knottin may be engineered into a viral envelope glycoprotein, for example, in an extracellular domain of such viral envelope glycoprotein. In one embodiment, the virus is a measles virus and the knottin is engineered to be present at the carboxyl terminus of the H protein, for example as a C-terminal extension of the H protein. In one embodiment, the virus is a herpes virus and the knottin moiety is engineered to be present at the N-terminus of glycoprotein D. In one embodiment, the virus is a lentivirus and the knottin moiety is present at the N-terminus of glycoprotein 120 or glycoprotein 141. In one embodiment, the virus is an adenovirus, and the knottin moiety is incorporated into the adenoviral fiber protein, for example, in the HI loop of the fiber, the C terminus of the fiber, or the knottin may be incorporated in the L1 loop of the hexon, the RGD loop in the penton base, or in the minor capsid protein IX. In one embodiment, the virus is a herpes simplex virus and the knottin is inserted into the gC and/or the gD protein. In one embodiment, the virus is gamma herpes virus saimiri and the native binding region of the viral glycoprotein ORF51 to is replaced with the knottin. In one embodiment, the virus is an adeno-associated virus and the knottin is incorporated into any of the six putative loops of the AAV2 capsid protein. In one embodiment, the virus is vaccinia virus strain IHD-J and the knottin is incorporated at the N-terminus of the nonessential hemagglutinin HA protein.

The scope of the invention extends to alternative methods of transductional targeting as well. For example, in one embodiment, the viral retargeting is achieved by incorporation of a scaffold moiety into the virus, the scaffold moiety being an attachment site for exogenously provided knottins, for example by functionalizing the knottin with biotin or avidin for conjugation to the scaffold moiety on the virus. Exemplary methods of altering viral tropism with scaffolds are described, for example, in U.S. Pat. No. 7,456,008, by Lindholm et al., entitled “Modified virus comprising one or more non-native polypeptides” and U.S. Pat. No. 6,261,554, by Valero, entitled “Compositions for Targeted Gene Delivery.”

In a related alternative embodiment, the use of “bispecific targeting moieties” is employed, wherein the targeting knottin is present in a fusion protein which further comprises a moiety that binds to the virus. Co-expression of the virus and the fusion protein results in a virus functionalized with the knottin. Exemplary methods of engineering viral tropism with bispecific moieties are described, for example, in U.S. Pat. No. 6,524,572, by Li, entitled “Targeting recombinant virus with a bispecific fusion protein ligand in coupling with an antibody to cells for gene therapy,” and United States Patent Application Publication Number 20030092068, by Itoh et al., entitled “Agents for adsorption and bridging for adenovirus.”

Cytotoxic Viruses. In one aspect, the scope of the invention encompasses a cytotoxic virus which preferentially infects target cells by means of the selected knottin moiety, and which kills the infected cells by viral lysis or other means. The target cell may be any cell type.

The cytotoxic viruses of the invention may kill target cells by infection and lysis by native viral proteins. Additionally, the cytotoxic virus may be augmented to express cytotoxic “suicide genes,” such as thymidine kinase, cytosine deaminase, rat cytochrome P450 or other cytotoxic genes known in the art, the expression of which increases the effectiveness of target cell killing.

In one embodiment, the engineered virus of the invention is an oncolytic virus which preferentially or selectively targets cancer cells. Various oncolytic viruses are known in the art and have been successfully tested as vectors to kill tumor cells. However, prior art oncolytic viruses have various shortcomings, including, for example, lack of selectivity for target cells and low infection rates. The engineered viruses described herein provide the art with a novel means of targeting oncolytic viruses to cancer cells with high specificity for the target, high rates of infection, and effective tumor killing. As used herein, “cancer cells” will include cells of tumors and other neoplastic conditions known in the art, for example, cells of glioblastoma, medulloblastoma, intraparenchymal atypical teratoid/rhabdoid tumors, breast cancer, ovarian cancer, prostate cancer, liver cancer, lung cancer, kidney cancer, melanoma, and any other cancer known in the art. Cancer cells, as used herein, will further encompass cells associated with tumors such as cells present in the tumor microenvironment, extracellular matrix, or tumor immune compartment.

In oncolytic implementations, the knottin's target species is a cancer cell target, which is overexpressed or differentially expressed by cancer cells or in the tumor microenvironment, for example a membrane protein. In one embodiment, the cancer target is selected from the group consisting of EGFR (ErbB-1), HER2 (ErbB-2), EphA2, HGFR (cMET), IGF-1 receptor (IGF-1R), C-X-C chemokine receptor-4 (CXCR4), protease-activated receptor (PAR)-1, follicle-stimulating hormone receptor (FSH-R), glucose-regulated protein (GRP), procaspase activating compound (PAC)-1, carcinoembryonic antigen (CEA), CEACAM5, CEACAM6, EpCAM (epithelial cell adhesion molecule), E-cadherin, EpCAM, glutamate carboxypeptidase 2, aminopeptidase N, also known as CD13, seprase or fibroblast activation protein (FAP-α), P Matriptase (membrane-type serine protease 1, MT-SP1), membrane type-1 matrix metalloproteinase (MT1-MMP)/MMP14, ADAM12, carbonic anhydrase nine (CAIX), GLUT, ABC, SLC5a, epithelial membrane antigen (EMA), also known as mucin-1 (MUC-1), extracellular matrix metalloproteinase inducer (EMMPRIN), also named basigin or CD147, GRP78, SCL1A7, and endoglin (CD105).

In one embodiment, the target species is an integrin. Integrins are cell surface receptors involved in cell motility, adhesion, and in the case of cancer cells, invasive processes. Some integrins are known to be overexpressed, relative to wild type cells, by cancer cells. Advantageously, the inventors of the present disclosure have determined that the higher abundance (compared to normal host cells) of integrins found on many kinds of tumor cells provides a basis for selective infection and effective killing of tumors by the engineered virus of the invention. In measles virus, the relative receptor threshold effect is that the more surface receptor present on a cell, the more efficacious is measles virus infection. Importantly, there is a threshold level of target binding moiety below which productive infection does not occur. For example, the αvβ₃, αvβ₅ and α₅β₁ integrins are expressed at low levels on normal cell types such as dendritic cells, T-lymphocytes, and vascular smooth muscle cells, however, the oncolytic virus of the invention does not infect these cells, as the level of integrin expression for these normal cells is too low to support productive infection.

In one embodiment, the engineered virus of the invention comprises a knottin that will bind to an integrin selected from the group consisting of αvβ3, αvβ5, αvβ6 α4β1, α5β1, and α6β4. In one embodiment, the knottin is SEQ ID NO: 1. In one embodiment, the knottin is selected from the group consisting of SEQ ID NO: 2 through SEQ ID NO: 22. These knottins were previously developed and described by others in Kimura et al., (2009). Engineered cystine knot peptides that bind alphavbeta3, alphavbeta5, and alpha5beta1 integrins with low-nanomolar affinity. Proteins 77: 359-369. It will be understood that the scope of the invention encompasses variants of the enumerated sequences, including variants comprising truncated versions of the sequences, variants with additional amino acids, variants having one or more amino acid substitutions, including substitutions with non-natural amino acids or amino acid analogs. The scope of the invention extends to variants having 70% or greater, 75% or greater, 80% or greater, 85% or greater, 90% or greater, or 95% or greater sequence identity to the enumerated sequences of SEQ ID NO: 1 through SEQ ID NO: 22.

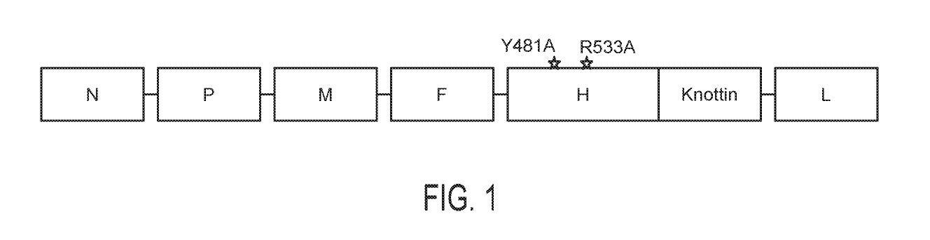

In one embodiment, the oncolytic virus is a measles virus. An exemplary measles virus construct is depicted in FIG. 1. In one embodiment, the measles virus is an Edmonston measles virus. In one embodiment, the oncolytic virus expresses an integrin-targeting knottin. In one embodiment, the integrin targeting knottin comprises a knottin selected from the group consisting of SEQ ID NO: 1 through SEQ ID NO: 22. In one embodiment, the knottin is SEQ ID NO: 1.

Transformation Vectors. The selectivity and high affinity for target species afforded by knottins provides a means of efficiently transforming target cells with viral vectors. Accordingly, in some embodiments, the engineered virus of the invention comprises a viral transformation vector, wherein the virus is configured to deliver one or more gene constructs to the target cell. In this implementation, the engineered virus of the invention comprises (1) a knottin which directs the virus to infect a particular type or class of target cells and (2) one or more nucleic acid constructs that is expressed in the target cell. The one or more nucleic acid constructs will comprise a nucleic acid sequence coding for one or more transgenes, as well as regulatory sequences which enable the integration and/or expression of the transgene(s) in the target cell. In the case of viruses that are normally cytotoxic, the transformation vector engineered virus of the invention will be modified such that it does not lyse and kill the target cells.

Nucleic Acid Constructs. The scope of the invention encompasses the various engineered viruses disclosed above. The scope of the invention further encompasses nucleic acid constructs coding for such engineered viruses, or portions thereof. The nucleic acids of the invention include DNA, RNA, and other nucleic acid sequences, including plasmids, expression vectors, and engineered viral genomes. The scope of the invention further extends to transformed cells carrying or expressing such nucleic acid sequences.

Methods of the Invention. In addition to the various compositions of matter disclosed above, the scope of the invention encompasses methods of using knottin-targeted viruses. In the general method of the invention, the method comprises the administration to an organism of an engineered virus, wherein the engineered virus comprises one or more knottin moieties, wherein the engineered virus selectively or preferentially infects a target cell type in the organism, wherein the target cell type expresses or displays a target binding moiety that is selectively bound by the one or more knottin moieties present in the engineered virus. The target cell may comprise any cell type, including, for example, cancer cells, stem cells, immune cells, and cells of the eye, brain, liver, kidney, lung, pancreas, muscle, or bone marrow.

Such administration may be to any organism. In some embodiments, the organism is an animal species. In some embodiments, the organism is a human, for example, a human patient in need of treatment for a pathological condition. In some embodiments, the organism is an animal species, for example a rat, mouse, horse, cow, dog, cat, pig, non-human primate, or animal of another species, for example, being treated in a veterinary or research context.

In the therapeutic context, the administration of the engineered virus encompasses the administration of a therapeutically effective amount of the engineered virus. For example, virus can be delivered at dosages of 10⁸ to 10¹² virus particles. The administration of engineered virus may be carried out using any applicable procedure for viral delivery, including by intravenous, intra-tumoral, intraperitoneal, intravascular, topical, aerosol, or other delivery mechanism. Such administration includes the co-administration of excipients, carriers, and other ancillary components necessary or desirable for viral delivery, as known in the art.

In an alternative embodiment, the engineered virus is administered ex-vivo, to explants or cultured cells. For example, in one embodiment, the virus is administered to cultured cells in order to transform them. In one embodiment, the virus is administered to cultured cells to selectively ablate target cells types, for example in tissue engineering applications.

Cytotoxic and Oncolytic Methods. In one embodiment, the method of the invention comprises the administration of a knottin-targeted virus to a subject for the selective killing of target cells.

In one embodiment, the invention comprises a method of treating cancer in an animal by the use of an oncolytic virus expressing one or more knottins, wherein the one or more knottins selectively bind to one or more tumor antigens. In one embodiment, the tumor antigen is an integrin.

In one embodiment, the invention comprises a virus expressing one or more knottins that bind integrin αvβ3, and further includes a method of using such virus to treat cancer, including melanoma, breast cancer, prostate cancer, pancreatic cancer, ovarian cancer, cervical cancer, or glioblastoma. In one embodiment, the invention comprises a virus expressing one or more knottins that bind integrin αvβ35, and further includes a method of using such virus to treat cancer, including melanoma or glioblastoma. In one embodiment, the invention comprises a virus that expresses one or more knottins that bind integrin αvβ6, and further includes a method of using such virus to treat cancer, including cervical cancer or colon cancer. In one embodiment, the invention comprises a virus expressing or more knottins that bind integrin α4β1, and further includes a method of using such virus to treat cancer, including ovarian cancer. In one embodiment, the invention comprises a virus expressing or more knottins that bind integrin α5β1, and further includes a method of using such virus to treat cancer, including non-small-cell lung carcinoma. In one embodiment, the invention comprises a virus expressing or more knottins that bind integrin α6β4, and further includes a method of using such virus to treat cancer, including breast cancer.

Gene Therapy Methods. In another aspect, the engineered viruses of the invention may be used to selectively transform target cells. In one embodiment, the method comprises a gene therapy method, for example, for the replacement or suppression of defective genes, genetic augmentation, or any other delivery of genetic material to a cell, including for integration into the target cell genome. The gene therapy construct may comprise any transgene, including those coding for enzymes, structural proteins, transcription factors, and others.

Immunotherapy Methods In another aspect, the methods of the invention encompass the use of the knottin-targeted viruses in an immunotherapy context.

In one implementation, the engineered virus comprises a cytotoxic virus which selectively targets a ligand presented by a specific type of immune cell, such that the target cell is infected and destroyed. For example, the target cell may comprise an immunosuppressive cell in the tumor microenvironment, wherein the destruction of such cells enhances the immune response against the tumor. Alternatively, the target cell may comprise a cell which is implicated in an autoimmune response, wherein the destruction of such cells reduces aberrant immune system activity.

In an alternative implementation, immunotherapy method comprises the administration of an engineered virus comprising a transformation vector, wherein a genetic construct is delivered to a particular subset of immune cells to modulate the immune activity of the target cells. For example, the delivered protein may comprise a cytokine, interleukin, chemokine, or other immune-modulating protein, for example to prime immune cells for tumor destruction or to inhibit an inflammatory or autoimmune response.

### EXAMPLE 1

### Retargeting of Oncolytic Measles Virus for Tumor-Associated Antigens

Introduction: In this example, a retargeted measles virus is demonstrated, comprising a knottin targeted to integrins (SEQ ID NO: 1), the virus being referred to herein as MV-CKPint. SEQ ID NO: 1 comprises the integrin binding motif arginine-glycine-aspartic acid (RGD). This knottin was specifically selected for restricted binding to the tumor specific integrins αᵥβ₃, αᵥβ₅, and α₅β₁, and binds to these integrins with single digit nanomolar affinity. In this example, it is demonstrated that measles virus can be retargeted to these integrins by the knottin and that MV-CKPint binds to, replicates in, and kills tumor cells in vitro. Proteolytic removal of the knottin targeting moiety abrogates infection and cell killing indicating that specific binding of the knottin motif mediates cellular entry of the virus. In addition, it is shown that this retargeted virus infects tumor cells in a murine xenograft model when delivered by intravascular injection. These results demonstrate the use of knottins to retarget measles virus or any other virus, especially an enveloped virus.

Results: The integrin-binding knottin motif is expressed at the c-terminus of the H-protein: Measles virus normally binds to its receptors via the H protein. The H-protein encoded by the viral genome used to manufacture MV-CKPint has two mutations (Y481A and R533A) that eliminate binding to CD46 and SLAM, the normal measles virus receptors. The integrin-binding knottin was expressed at the carboxyl end of the H-protein, resulting in its exposure on the surface of the virus. The MV-CKPint virus was modeled after basic viral construct depicted in FIG. 1. MV-CKPint also expressed a 6× HisTag at the C-terminus of the H protein (after the knottin moiety) and a GFP moiety fused to n-terminus of the nucleoprotein. The functionality of the MV-CKPint was verified by infecting αHis-vero cells. These modified cells express membrane-bound single chain antibody for hexahistidine, hence facilitate viral entry through binding of the 6× His tag at the c-terminus of the MV-CKPint virus. The virus was also tested on the D283-medulloblastoma cell line. The MV-CKPint virus successfully infected αHis-vero and D283-med cells, and large syncytia fluorescing with the GFP were formed in both cell lines. In D283-med cells, GFP florescence is clearly visible in the perinuclear space, which is typical for measles virus. This finding indicates that adding the integrin-binding knottin to the H protein has no adverse effect on the infection or the replication of the virus.

MV-CKPint infects and produces cytopathic effect in cells of different tumor type: To investigate knottin-mediated cellular infection, tumor cell lines of different origin, glioblastoma, breast carcinoma, diffuse intrinsic pontine glioma (DIPG), and medulloblastoma, were analyzed for expression of target integrins. Flow cytometry confirmed that all tested cell lines expressed αvβ₃, αvβ₅, and α₅β₁ integrins at high levels on the membrane. The MV-CKPint virus contains a GFP coding sequence, so GFP florescence is a marker of infection. After infection with the virus, cell lines of all tested tumor types showed multiple green florescent syncytia. Syncytia formation became visible as early as 48 hrs post infection. The formation of syncytia indicate that MV-CKPint efficiently infects cells expressing target integrins.

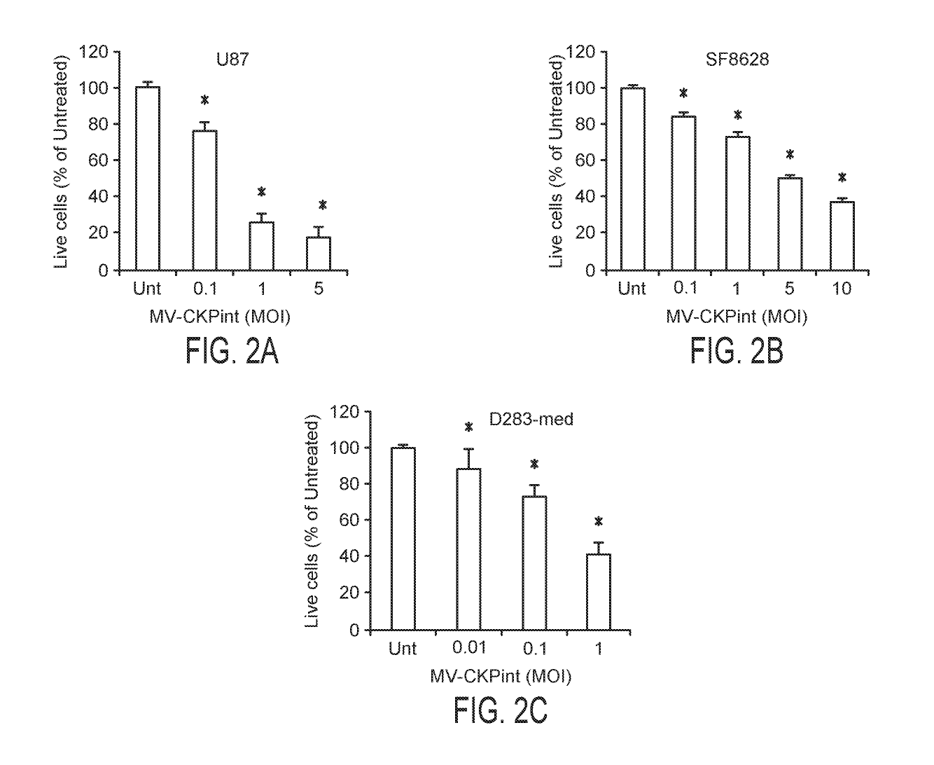

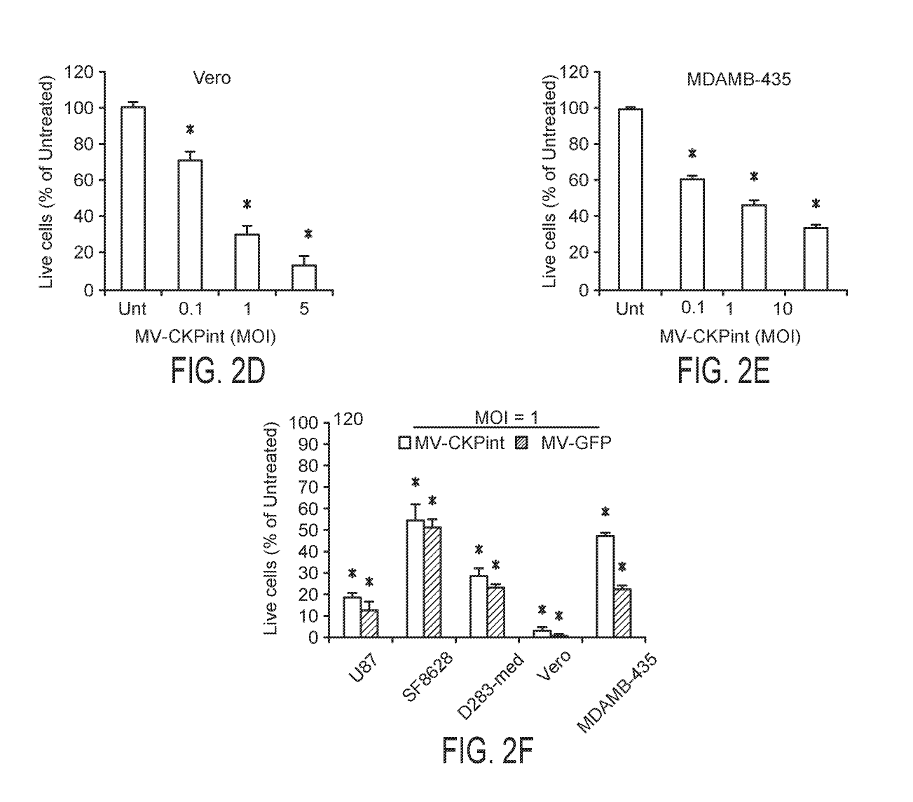

MV-CKPint replicates in and kills cells of different tumor types: Glioblastoma, breast carcinoma, DIPG, medulloblastoma and Vero cells were infected with increasing MOI of MV-CKPint virus. The virus killed all of these cell types in dose and time-dependent manner as depicted in FIGS. 2A, 2B, 2C, and 2D. Since the level of target integrin dimers varies on different cell types, the oncolytic potential of the virus also varied for different tumor types. When infected at MOI of 1, 75% glioblastoma cells and 60% medulloblastoma cells died after 96 h, whereas, infection at MOI of 5 was needed to kill 50% DIPG tumor cells in that period.

To determine the replication competency of MV-CKPint, human glioblastoma and breast carcinoma cells were infected with the virus, and the titers of cell-associated virus were quantified daily after infection. The titer of infectious virus particles in these cells increased with time over the period of 4 days post infection. Since MV-CKPint contains GFP as a marker, the replication of the virus in live infected cells was also measured by flow cytometry. The intensity of GFP florescence in infected glioblastoma and breast cancer cells increased over time. Thus, MV-CKPint both replicates in and kills tumor cells in vitro.

Infection of MV-CKPint is dependent on the integrin binding of the knottin: To confirm the integrin-binding motif of the knottin is responsible for cell binding by MV-CKPint, the modified virus was assessed in the presence of echistatin, a potent competitive binder of αvβ₃, and α₅β₁ integrins. Human glioblastoma and breast cancer cells were infected with MV-CKPint in the absence and presence of soluble echistatin. Echistatin significantly decreased cell killing by MV-CKPint. Thus, echistatin binding to target integrins appears to prevent viral binding via the knottin.

To confirm integrin-binding-mediated cellular entry of the virus, the knottin motif was removed from the virus using FXa protease cleavage site present right before the knottin sequence in the viral genome. Pre-incubation of virus with FXa protease markedly abrogated cell killing in dose-dependent fashion. This further confirms that the integrin binding motif is required for cell binding and entry.

MV-CKPint reaches tumor sites and forms syncytia after intravenous delivery: To investigate intravenous (IV) delivery of this modified virus, mice bearing subcutaneous U87-MG glioblastoma tumors were injected with a single dose of MV-CKPint (0.5×10⁶ TCID₅₀/100 μl) or an equal volume of PBS (control) via the tail vein. Additional control mice were injected directly into the tumor with the same dose of MV-CKPint. After 8 days post injection, tumors were harvested and analyzed for the presence of measles virus by immunohistochemistry. In tumors from mice receiving IV injection of MV-CKPint, multiple scattered small syncytia were noticed. The syncytia stained positive with the measles virus nucleoprotein-specific antibody indicating for the presence of measles virus. For intratumoral (IT) injection, a large but localized region of measles virus nucleoprotein-positive syncytia was observed at the injection site. The adjacent sections from same tumors used for IHC were also examined by immunofluorescence staining to confirm the presence of measles virus. This indicates that MV-CKPint virus successfully reached the tumor bed after IV injection.

Discussion: In the treatment of solid tumors, measles virus has required intratumoral injection for efficacy. Indeed, both the GBM and the medulloblastoma phase 1 trials for solid tumor recurrence use direct injection of the tumor resection bed for virus delivery. Because the αvβ₃, αvβ₅, and α₅β₁ integrins targeted by the knottin used here have been shown to be expressed by tumor vascular endothelium, the use of this knottin for therapy with IV injection of virus was tested. Animals bearing flank tumors were injected with a single dose of 5×10⁵ TDIC₅₀ of MV-CKPint intravenously and tumors were harvested for analysis at 8 days after injection. A localized large area of syncytia was observed in IT-treated tumors compared to multiple small foci of infection with intravascular virus delivery. The successful delivery of MV-CKPint in xenograft tumors upon IV injection suggests that the virus is infecting and replicating in tumor vascular endothelium and the resultant virus is then spreading to the tumor cells.

An issue with the use of knottins for virus targeting is the requirement for correct peptide folding and correct formation of the disulfide bonds in the knottin for binding activity. It appears that passage of the viral protein containing the knottin through an infected cell's endoplasmic reticulum provides the proper environment to assure proper folding and bond formation of the knottin.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. An engineered virus comprising,
one or more knottin polypeptides, wherein the one or more knottin polypeptides is configured such that it confers selective infection of a target cell type by the virus,
wherein the target cell type expresses one or more target binding moieties that is selectively bound by the one or more knottins.

2. The engineered virus of claim 1, wherein
the virus is an enveloped virus.

3. The engineered virus of claim 2, wherein
the enveloped virus is a virus selected from the group consisting of measles virus, herpes virus, lentivirus, alpha virus, pox virus, and vaccinia virus.

4. The engineered virus of claim 1, wherein
the virus is a cytotoxic virus, and wherein the virus kills the infected target cells.

5. The cytotoxic virus of claim 4, wherein
the virus further comprises one or more cytotoxic genes.

6. The cytotoxic virus of claim 4, wherein
the virus is an oncolytic virus and the target cell is a cancer cell.

7. The cytotoxic virus of claim 6, wherein
the target binding moiety is an integrin.

8. The cytotoxic virus of claim 7, wherein
the one or more knottins comprises a polypeptide selected from the group consisting of SEQ ID NO: 1-SEQ ID NO: 22, or a variant thereof.

9. The cytotoxic virus of claim 8, wherein
the one or more knottins comprises SEQ ID NO: 1.

10. The engineered virus of claim 1, wherein
The virus is a transformation vector comprising one or more nucleic acid constructs that are expressed in infected target cells.

11. A method of selectively infecting a target cell, comprising
administering an engineered virus to the target cell wherein the engineered virus comprises one or more knottin polypeptides which selectively bind to one or more target binding moieties present on the target cell.

12. The method of claim 11, wherein
the virus is administered in a pharmaceutically effective amount to a human patient in need of treatment for a pathological condition.

13. The method of claim 11, wherein
the engineered virus is a cytotoxic virus and infection of the target cells results in the killing of the infected cells.

14. The method of claim 13, wherein
the engineered virus is an oncolytic virus and the target cells are cancer cells.

15. The method of claim 14, wherein
the one or more knottin polypeptides selectively binds to an integrin.

16. The method of claim 15, wherein
the knottin is a polypeptide selected from the group consisting of SEQ ID NO: 1-SEQ ID NO: 22, or a variant thereof.

17. The method of claim 14, wherein
the engineered virus is a enveloped virus.

18. The method of claim 11, wherein
the engineered virus is a transformation vector which delivers one or more nucleic acid constructs to the target cell and wherein the one or more nucleic acid constructs is expressed by infected target cells.

19. The method of claim 18, wherein
the one or more nucleic acid constructs is a gene therapy construct.

20. The method of claim 11, wherein
the target cell is an immune cell and infection of the cell by the engineered virus results in the death of the target cell or the modulation of immune activity in the target cell.


SEQUENCE LISTING (22 sequences)

SEQ ID NO: 1 - protein, 33 aa, Artificial Sequence
  Description of Artificial Sequence Synthetic polypeptide
GCPRPRGDNPPLTCKQDSDCLAGCVCGPNGFCG

SEQ ID NO: 2 - protein, 33 aa, Artificial Sequence
  Description of Artificial Sequence Synthetic polypeptide
GCPEPRGDNPPPSCKQDSDCRAGCVCGPNGFCG

SEQ ID NO: 3 - protein, 33 aa, Artificial Sequence
  Description of Artificial Sequence Synthetic polypeptide
GCLPPRGDNPPPSCKQDSDCQAGCVCGPNGFCG

SEQ ID NO: 4 - protein, 33 aa, Artificial Sequence
  Description of Artificial Sequence Synthetic polypeptide
GCHLGRGDWAPVGCKQDSDCPAGCVCGPNGFCG

SEQ ID NO: 5 - protein, 33 aa, Artificial Sequence
  Description of Artificial Sequence Synthetic polypeptide
GCNVGRGDWAPSECKQDSDCPAGCVCGPNGFCG

SEQ ID NO: 6 - protein, 33 aa, Artificial Sequence
  Description of Artificial Sequence Synthetic polypeptide
GCFPGRGDWAPSSCKQDSDCRAGCVCGPNGFCG

SEQ ID NO: 7 - protein, 33 aa, Artificial Sequence
  Description of Artificial Sequence Synthetic polypeptide
GCPLPRGDNPPTECKQDSDCQAGCVCGPNGFCG

SEQ ID NO: 8 - protein, 33 aa, Artificial Sequence
  Description of Artificial Sequence Synthetic polypeptide
GCSEARGDNPRLSCKQDSDCRAGCVCGPNGFCG

SEQ ID NO: 9 - protein, 33 aa, Artificial Sequence
  Description of Artificial Sequence Synthetic polypeptide
GCLLGRGDWAPEACKQDSDCRAGCVCGPNGFCG

SEQ ID NO: 10 - protein, 33 aa, Artificial Sequence
  Description of Artificial Sequence Synthetic polypeptide
GCHVGRGDWAPLKCKQDSDCQAGCVCGPNGFCG

SEQ ID NO: 11 - protein, 33 aa, Artificial Sequence
  Description of Artificial Sequence Synthetic polypeptide
GCVRGRGDWAPPSCKQDSDCPAGCVCGPNGFCG

SEQ ID NO: 12 - protein, 33 aa, Artificial Sequence
  Description of Artificial Sequence Synthetic polypeptide
GCLGGRGDWAPPACKQDSDCRAGCVCGPNGFCG

SEQ ID NO: 13 - protein, 33 aa, Artificial Sequence
  Description of Artificial Sequence Synthetic polypeptide
GCFVGRGDWAPLTCKQDSDCQAGCVCGPNGFCG

SEQ ID NO: 14 - protein, 33 aa, Artificial Sequence
  Description of Artificial Sequence Synthetic polypeptide
GCPVGRGDWSPASCKQDSDCRAGCVCGPNGFCG

SEQ ID NO: 15 - protein, 33 aa, Artificial Sequence
  Description of Artificial Sequence Synthetic polypeptide
GCYQGRGDWSPSSCKQDSDCPAGCVCGPNGFCG

SEQ ID NO: 16 - protein, 33 aa, Artificial Sequence
  Description of Artificial Sequence Synthetic polypeptide
GCAPGRGDWAPSECKQDSDCQAGCVCGPNGFCG

SEQ ID NO: 17 - protein, 33 aa, Artificial Sequence
  Description of Artificial Sequence Synthetic polypeptide
GCVQGRGDWSPPSCKQDSDCPAGCVCGPNGFCG

SEQ ID NO: 18 - protein, 33 aa, Artificial Sequence
  Description of Artificial Sequence Synthetic polypeptide
GCHVGRGDWAPEECKQDSDCQAGCVCGPNGFCG

SEQ ID NO: 19 - protein, 33 aa, Artificial Sequence
  Description of Artificial Sequence Synthetic polypeptide
GCDGGRGDWAPPACKQDSDCRAGCVCGPNGFCG

SEQ ID NO: 20 - protein, 33 aa, Artificial Sequence
  Description of Artificial Sequence Synthetic polypeptide
GCPQGRGDWAPTSCKQDSDCRAGCVCGPNGFCG

SEQ ID NO: 21 - protein, 33 aa, Artificial Sequence
  Description of Artificial Sequence Synthetic polypeptide
GCPQGRGDWAPEWCKQDSDCPAGCVCGPNGFCG

SEQ ID NO: 22 - protein, 33 aa, Artificial Sequence
  Description of Artificial Sequence Synthetic polypeptide
GCPRGRGDWSPPACKQDSDCQAGCVCGPNGFCG